(19) United States
(12) Patent Application Publication
(10) Pub. No.: US 2016/0051585 A1
(43) Pub. Date: Feb. 25, 2016

(54) COMBINED PROTEIN SUPPLEMENTS

(71) Applicant: 4LIFE PATENTS, LLC, Sandy, UT (US)

(72) Inventor: Christopher M. Lockwood, Draper, UT (US)

(21) Appl. No.: 14/698,698

(22) Filed: Apr. 28, 2015

Related U.S. Application Data
(60) Provisional application No. 61/985,277, filed on Apr. 28, 2014.

(51) Int. Cl. A61K 35/20; A61K 35/57; A23J 3/00; A23L 1/29; A23L 1/305; A61K 38/17; A61K 9/00

(52) U.S. Cl. CPC A61K 35/20; A61K 38/1709; A61K 38/1703; A61K 35/57; A61K 9/0053; A23L 1/296; A23L 1/3053; A23J 3/04; A23V 2002/00

(57) ABSTRACT

Protein supplements that include proteins from animal products, such as whey and eggs, are disclosed. Such a protein supplement may also include one or more immune modulators, such as transfer factor and/or nanofraction immune modulators.

### CROSS-REFERENCE TO RELATED APPLICATION

A claim to the benefit of the Apr. 28, 2015 filing date of U.S. Provisional Patent Application No. 61/985,277, and titled COMBINED PROTEIN SUPPLEMENTS (“the '277 Provisional Application”) is hereby made pursuant to 35 U.S.C. §119(e). The entire disclosure of the '277 Provisional Application is hereby incorporated by reference.

### TECHNICAL FIELD

This disclosure relates generally to protein supplementation and, more specifically to protein supplements that include proteins from animal products, such as whey and eggs. This disclosure also relates to protein supplements that include immune modulators, such as transfer factor and/or nanofraction immune modulators.

### RELATED ART

Protein supplementation has long been used in connection physical activities, including athletic training, resistance training (e.g., weight training, etc.) and other types of exercise. Athletes and other individuals who subject their bodies to intense and prolonged physical exercise often require more protein than their diets provide. They typically obtain additional protein from a protein supplement, such as a protein shake (usually made by mixing a protein powder into a drinkable liquid, such as water, a dairy product or a juice), a protein bar or a wide variety of other edible products. The added protein intake is believed to contribute to muscle growth (hypertrophy), or increases in muscle mass.

Because of the well-publicized use of protein supplements by professional athletes—particularly weightlifters and others who undergo intensive resistance training regimens, the use of protein supplements has also become widespread amongst normal individuals in connection with their workouts. While protein supplementation is most commonly used by individuals who train with weights or other resistance equipment or who perform other types of resistance training, many people consume protein supplements in connection with endurance training and other aerobic activities.

Conventional sources of protein supplementation have included whey and other milk products (e.g., casein protein, etc.), eggs and soybeans. Soy-based protein products are widely used due, at least in part, to their low cost relative to egg-based protein products and milk-based protein products. Whey-based protein products are, however, believed to provide greater benefits (i.e., increases in muscle mass) than soy-based protein products.

### SUMMARY

Protein supplements that include (or even consist essentially of or consist of) protein from animal products are disclosed. A protein supplement according to this disclosure may be embodied as a protein powder, which may be configured for incorporation into (e.g., blending with, etc.) another edible product. Some non-limiting examples of edible products include drinks (e.g., water, milk, juice, etc.), baby formulas (in which the protein powder may replace at least some of the proteins in the baby formula), an energy bar, a meal replacement bar, a chew, a gel, a ready-to-drink (RTD) shake, a liquid, another food or the like.

In one aspect, a protein supplement may include two or more sources of protein that are obtained from animals and that are accordingly referred to herein as “animal protein sources.” The protein supplement may lack or substantially lack proteins from soybeans, or so-called “soy proteins,” or other proteins from vegetative (i.e., non-animal) sources. Such a protein supplement may consist essentially of animal protein sources and their respective proteins, or even consist of the animal protein sources. Some examples of suitable animal protein sources include milk and milk products (e.g., whey, casein, etc.) and eggs and egg products.

In some embodiments the animal protein source may comprise an intact source, which has not been concentrated, subjected to isolation processing or subjected to hydrolytic processing. In other embodiments, the animal protein source may comprise a concentrate or an isolate. Alternatively, a hydrolysate of an intact animal protein source, an animal protein source concentrate or an animal protein source isolate may be used in the protein supplement.

In another aspect, a protein supplement may include a protein source and an immune modulator. The protein supplement may lack or substantially lack proteins from soybeans, or so-called “soy proteins,” or other proteins from vegetative (i.e., non-animal) sources. In some embodiments, the protein may consist essentially of or even consist of one or more animal protein sources.

In embodiments where a protein supplement includes an immune modulator, the immune modulator may comprise one or more low molecular weight immune modulating molecules, such as transfer factor, low molecular weight immune modulating molecules that are larger than transfer factor, nanofraction immune modulators (which are smaller than transfer factor), or combinations of thereof. The source of an immune modulator may comprise colostrum, egg, egg yolk, a fraction of any of the foregoing, or any combination of the foregoing. Fractions of sources may, without limitation, comprise fractions that are defined by any of a variety of filters or dialysis membranes that provide a desired molecular weight cutoff (MWCO). As those of ordinary skill in the art will appreciate, a MWCO may refer to a molecular weight that defines an average cutoff for the materials that pass through a filter or dialysis membrane, with some smaller molecules being trapped by the filter or dialysis membrane and some larger molecules passing through the filter or dialysis membrane. When a source of immune modulators is subjected to filtering with a 12 KD (kiloDalton) filter, the filtrate or dialysate will include transfer factor, as well as immune modulators that are larger than transfer factor (e.g., in the range of about 8 KD to about 12 KD), transfer factor (including molecules of about 4 KD to about 6 KD) and nanofraction immune modulators (e.g., immune modulators having molecular weights of less than 3 KD, less than 2 KD, between about 250 D and about 2 KD, etc.). A filter or dialysis membrane that has a MWCO of 10 KD may be used to obtain a filtrate or dialysate that includes transfer factor and nanofraction immune modulators. A filtrate or dialysate with a MWCO of 5 KD will include nanofraction molecules.

In a specific embodiment, a protein supplement may consist essentially of a whey protein hydrolysate, a colostrum fraction having a molecular weight cutoff of 10 KD and a colostrum fraction having an upper MWCO of 5 KD. In another specific embodiment, a protein supplement may consist essentially of a whey protein hydrolysate, egg yolk, a colostrum fraction having an upper MWCO of 10 KD and a colostrum fraction having an upper MWCO of 5 KD.

Other aspects of the disclosed subject matter include edible products with embodiments of protein supplements according to this disclosure. Various embodiments of edible products that include protein supplements include, but are not limited to, baby formulas (because low molecular weight equates to low allergenicity), protein bars, meal replacement bars, chews, gels, ready-to-drink shakes, liquids, and other foods.

Methods in which a protein supplement according to this disclosure is administered to an individual are also disclosed. In one embodiment, such a method includes consuming the protein supplement (e.g., blended with a liquid, as part of a food product, etc.) in the morning for breakfast. As another option, a protein supplement or a food product including the same may be consumed as a snack at least twice each day. A protein supplement may be taken in conjunction with one or more other supplements. Each of the foregoing methods may be effected alone, or they may be conducted in any combination. In addition, any of the foregoing methods or any combination of the foregoing methods may be combined with a regular, even progressive, exercise program, which may include resistance training, cardiovascular (i.e., aerobic) activity or a combination thereof.

Other aspects, as well as features and advantages of various aspects of the disclosed subject matter will become apparent to those of ordinary skill in the art through consideration of the ensuing description and the appended claims.

### BRIEF DESCRIPTION OF THE DRAWINGS

In the drawings:

FIGS. 1a-8b and 10a-11 provide graphic representations of the results of various analysis that were conducted to determine potential benefits of various embodiments of protein supplements; and

FIG. 9 provides graphical illustration of the mTORC1 pathway.

### DETAILED DESCRIPTION

A protein supplement according to this disclosure may include an animal protein source of protein, or any combination of animal protein sources. In some embodiments, one or more animal protein sources may be combined with an immune modulator.

The animal protein source may comprise a product obtained from an animal, such as a dairy product (e.g., whey, casein, milk, etc.), an egg-based product (e.g., whole egg, egg white, egg yolk, a fraction of an egg, etc.) or the like. Animal protein sources for proteins may be obtained from a variety of different animals. Without limitation, a dairy product may be obtained from a bovine source (e.g., from cows). Other sources of dairy products include, but are not limited to, goats, camels, yaks and other mammals. Egg products may be obtained from a variety of sources, including, without limitation, chickens, ostriches and other birds.

An animal protein source of protein may comprise an intact source of protein, which may be in a liquid, a semisolid or a powdered form. Some examples of intact sources of protein include whey powder, casein powder, egg albumin (i.e., egg white), which consists substantially of protein and requires little additional processing (e.g., de-sugaring, etc.) or no additional processing.

Alternatively, the animal protein source may comprise a concentrate, in which the protein concentration exceeds a protein concentration of an intact version of the same animal protein source. As another option, the animal protein source may comprise an isolate, which may include an even greater protein content than a concentrate.

To provide an example of the differences in protein content between intact animal protein sources, animal protein source concentrates and animal protein source isolates, intact whey may have a protein content of about 30%, by weight (w/w), while the protein content of a whey protein concentrate (WPC) may be as high as about 80% w/w and the protein content of a whey protein isolate may be as high as about 90% w/w.

As yet another option, the animal protein source of protein may comprise a hydrolysate, in which the proteins have been predigested to decrease their average size and to enhance their absorption by and incorporation into an individual's body. A hydrolysate may be prepared from an intact animal protein source, an animal protein source concentrate or an animal protein source isolate, and may have substantially the same protein content as the animal protein source had before it was subject to hydrolytic processing.

In embodiments where a protein supplement includes two or more animal protein sources, the animal protein sources may have different characteristics that make their combined use beneficial. As an example, an animal protein source from a first type of animal may include relatively high, or plentiful, amounts of some amino acids and relatively low amounts of, or be deficient in, one or more other amino acids. A second type of animal may produce animal protein sources that include plentiful amounts of the amino acids that are deficient in the animal protein source produced by the first animal. Thus, by combining animal protein sources from the first and second animals, the resulting mixture may include adequate amounts of a greater number of amino acids than would have been provided by either animal protein source alone.

Conversely, the amount of one or more amino acids in an animal protein source produced by a first animal may be undesirably high for administration to an individual. For example, while leucine increases protein synthesis by skeletal muscles and, thus, promotes muscle growth, and it may suppress degradation of proteins in skeletal muscle, undesirably high amounts of leucine may also lead to loss of appetite. Such an animal protein source may be combined with an animal protein source that includes less of (e.g., is deficient in, etc.) that (those) amino acids, effectively diluting the amount(s) of that (those) amino acids in the mixture, and possibly providing a mixture with adequate amounts of other amino acids. The resulting mixture may be safer to consume than the animal protein source from the first animal alone.

Substantially all of the protein of a protein supplement may be obtained from animal protein sources. Stated another way, a protein supplement may include substantially no protein from a non-animal protein source, for example, from a vegetative source, such as soybeans. In this context, the term “substantially” indicates that a protein supplement may include incidental amounts of non-animal proteins, such as those present in various other ingredients (e.g., plant-based ingredients, fungal extracts, colorants, flavorings, etc.). Thus, the protein of a protein supplement may consist essentially of protein from animal protein sources. In some embodiments, the protein of a protein supplement may even consist of protein from animal protein sources.

A variety of immune modulators may be used with a protein supplement. Some examples of suitable immune modulators, including, without limitation, transfer factor, low molecular weight immune modulators (e.g., immune modulating molecules having molecular weights in the range of about 8 KD to about 12 KD, etc.), such as those disclosed by U.S. Patent Application Publication No. 2008/0081076 of Lisonbee, et al. (hereinafter “Lisonbee”), the entire disclosure of which is hereby incorporated herein, and nanofraction immune modulators, such as those disclosed by Lisonbee.

Like the proteins of the protein supplement, the immune modulators may be obtained from animal sources, such as colostrum, eggs and other animal products. A protein supplement may include one or more immune modulators from a single type of source animal, or it may include immune modulators from a plurality of different types of source animals.

Different types of animals may be exposed to different types of antigens or pathogens, such as by vaccination, the environments in which the animals live or the like. Thus, when immune modulators from two or more different types of source animals are included in a protein supplement, the immune modulators and the protein supplement with which they are included may provide a broader array of antigen specificity or pathogen specificity than immune modulators from a single type of source animal. Consequently, a composition that includes immune modulators from two or more different types of source animals may be capable of enlisting the immune system of a treated animal to elicit a T-cell mediated immune response against a broader array of pathogens than those against which compositions that include immune modulators from a single source are effective.

By way of example, and not by way of limitation, a protein supplement may include immune modulators from both bovine sources (e.g., cows) and chickens. As cows may be exposed to different antigens or pathogens than those to which chickens are exposed, a protein supplement that includes both bovine-derived and chicken-derived sources will include a broader array of immune modulators than a protein supplement that only includes one or more bovine-derived sources of immune modulators or one or more chicken-derived sources of immune modulators. More specifically, a protein supplement with both a bovine-derived source and a chicken-derived source may include immune modulators that are specific to antigens or pathogens to which cows are exposed, as well as immune modulators that have specificity for antigens or pathogens to which chickens are exposed.

One or more sources of immune modulators of a protein supplement according to this disclosure may lack or substantially lack proteins and other molecules over a certain, predetermined size. A variety of processes may be used to select for smaller proteins and other molecules. Without limitation, filtration and dialysis processes may be used to impart a source of immune modulators with a desired upper molecular weight cutoff. Some examples of upper MWCOs for sources of immune modulators include about 12 KD, about 10 KD, about 8 KD, about 6 KD, about 5 KD, about 3 KD, about 2 KD and about 500 Da. See, e.g., Lisonbee, TABLE 1.

In embodiments where a protein supplement includes an immune modulator, the immune modulator may comprise one or more low molecular weight immune modulating molecules, such as transfer factor (e.g., fractions with upper MWCO's of about 12 KD, about 10 KD, about 8 KD and about 6 KD, provided that they do not have lower MWCOs above about 5 KD; etc.), nanofraction immune modulators (e.g., fractions with upper MWCO's of about 5 KD, about 4 KD, about 3 KD and about 2 KD, provided that they do not have lower MWCOs above about 2 KD; etc.) and other low molecular weight immune modulating molecules (e.g., fractions with upper MWCOs of about 12 KD and about 10 KD, etc.), or combinations of thereof.

The immune modulator may comprise colostrum, egg, egg yolk, a fraction of any of the foregoing, or any combination of the foregoing. Fractions may, without limitation, comprise fractions that are defined by any of a variety of filters that provide a desired molecular weight cutoff (MWCO). As those of ordinary skill in the art will appreciate, a MWCO may refer to a molecular weight that defines an average cutoff for the materials that pass through a filter, with some smaller molecules being trapped by the filter and some larger molecules passing through the filter. When a source of immune modulators is subjected to filtering with a 12 KD (kiloDalton) filter, the filtrate will include transfer factor, as well as immune modulators that are larger than transfer factor (e.g., in the range of about 8 KD to about 12 KD), transfer factor (including molecules of about 4 KD to about 6 KD) and nanofraction immune modulators (e.g., immune modulators having molecular weights of less than 3 KD, less than 2 KD, between about 250 D and about 2 KD, etc.). A filter that has a MWCO of 10 KD may be used to obtain a filtrate that includes transfer factor and nanofraction immune modulators. A filter with a MWCO of 5 KD will include nanofraction molecules.

In some embodiments, the sources for the immune modulators of a protein supplement may be the same as the animal protein sources for the protein of the animal supplement. As a non-limiting example, a protein supplement may include bovine whey as its animal protein source for protein and bovine colostrum or a fraction of bovine colostrum as its source for one or more types of immune modulators. As another non-limiting example, a protein supplement may include chicken eggs as its animal protein source and a fraction of chicken eggs as its source for immune modulators. In a specific embodiment, a protein supplement may consist essentially of a whey protein hydrolysate, a colostrum fraction having a molecular weight cutoff of 10 KD and a colostrum fraction having a MWCO of 5 KD.

In other embodiments, the sources for the immune modulators and the animal protein sources may be obtained from different types of animals. In a specific embodiment, a protein supplement may consist essentially of a whey protein hydrolysate, egg yolk, a colostrum fraction having a molecular weight cutoff of 10 KD and a colostrum fraction having a MWCO of 5 KD. Even more specifically, the egg yolk and the colostrum fractions may be provided as TRI-FACTOR® immune modulators, available from 4Life Research, LC of Sandy, Utah. TRI-FACTOR® immune modulators may include about 68% w/w of a bovine colostrum fraction having a molecular weight cutoff of 10 KD, about 2% w/w of a bovine colostrum fraction having an upper MWCO of 5 KD (with 80% or more of the proteins of the fraction, by weight of the fraction, having molecular weights of less than 5 KD and about 50% or more of the proteins of the fraction, by weight, having molecular weights of less than 3 KD) and about 30% w/w chicken egg yolk.

In specific embodiments, each dose of a protein supplement may include 10 g of the protein or combination of proteins and 100 mg of the immune modulator(s).

Specific embodiments of protein supplements that include two or more animal protein sources are set forth in the following example:

### Example

Powdered whey protein hydrolysate (WPH) was combined with a powdered egg white hydrolysate (EPH) in varying amounts, including: (i) about 70% w/w WPH and about 30% w/w EPH; (ii) about 50% w/w WPH and about 50% w/w EPH; and (iii) about 30% w/w WPH and about 70% w/w EPH.

### Additional Examples

Each of the three protein-immune modulator blends of the EXAMPLE provided above was tested to determine its effects on various markers of insulin signaling, skeletal muscle growth, or anabolism, skeletal muscle metabolism and adipose tissue lipolysis. The procedures and results of those studies follow, along with a discussion of the results:

### Insulin Signaling and Skeletal Muscle Anabolism

We examined how gavage-feeding the following dietary proteins (10 human equivalent grams dissolved in 1 ml of water) acutely affected skeletal muscle insulin signaling and anabolism markers 90- and 180-min post-feeding in male Wistar rats (˜250 g) fasted overnight.

Supplementation included: a) 80% whey protein concentrate (WPC, n=15); b) 70% hydrolyzed whey+30% hydrolyzed egg albumin (70W/30E, n=15); c) 50 W/50E (n=15); d) 30 W/70E (n=15); and e) 1 ml of water with no protein (fasted, n=14).

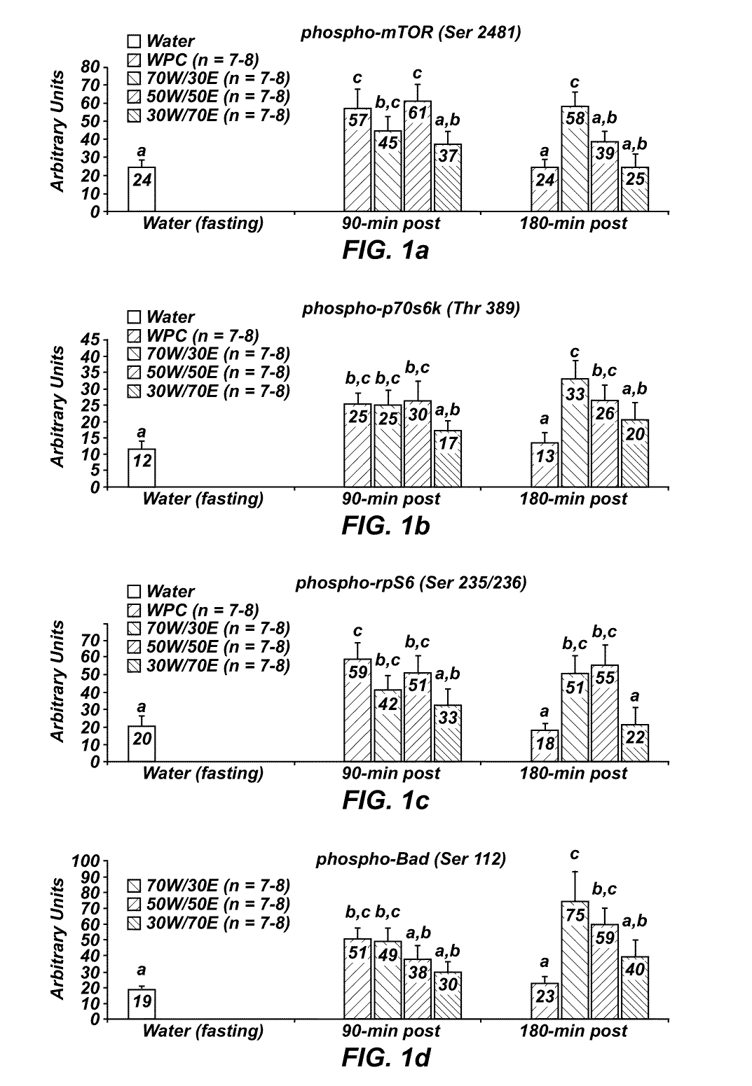

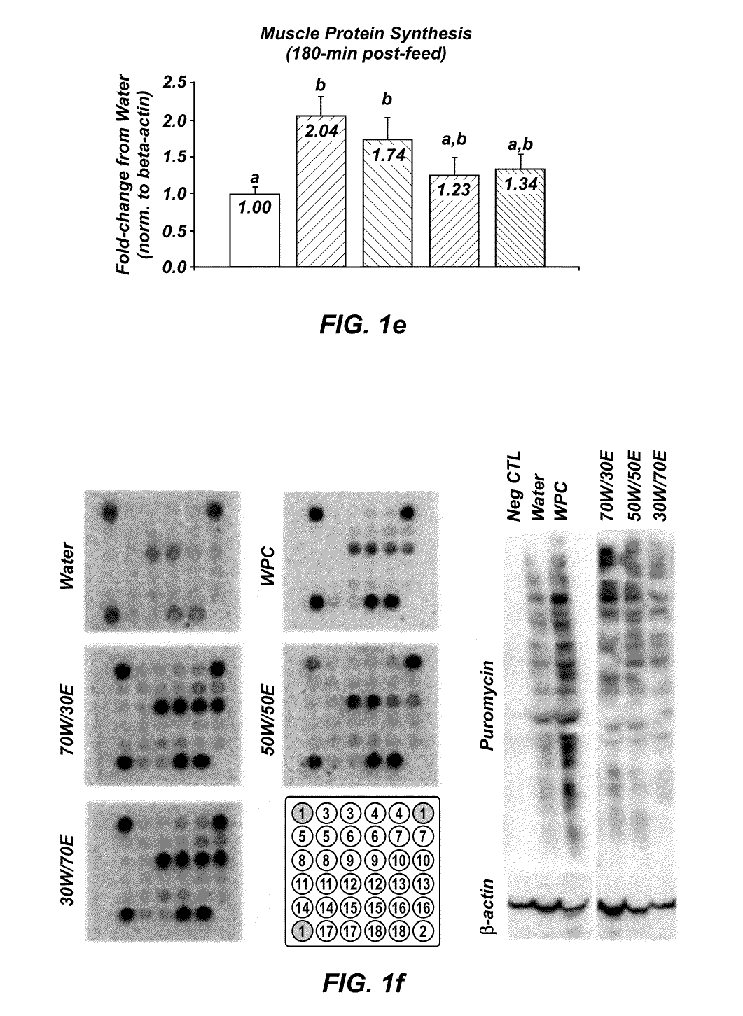

FIG. 1 presents the data obtained from testing. Data are presented in FIG. 1 as means±standard error (water n=12-14 per bar, protein feeding groups n=6-8 per bar). One-way ANOVA with LSD post-hoc analyses were performed and significant between-feeding differences are represented with different superscript letters (p<0.05). Panels a-d are anabolic (mTOR, p70s6k, rpS6) and anti-catabolic (Bad) phosphorylation markers. Panel e (left inset): Representative digital images of Akt-mTOR arrays of water and 180-min post protein feedings; key outlining phosphorylated targets—1: positive control (normalizer), 2: negative control, 3: p-Akt (Thr308), 4: p-Akt (Ser473), 5: p-rpS6 (Ser235/236), 6: p-AMPKα (Thr172), 7: p-PRAS40 (Thr246), 8: p-mTOR (Ser2481), 9: p-GSK-3α (Ser21), 10: p-GSK-3β (Ser9), 11: p-p70s6k (Thr389), 12&14: p-p70s6k (Thr421/Ser424), 13: p-Bad (Ser112), 15: p-PTEN (Ser380), 16: p-PDK1 (Ser241), 17: p-Erk1/2 (Thr202/Tyr204), 18: p-4E-BP1 (Thr37/46). Panel e (right inset): Representative digital images of puromycin integration into muscle protein.

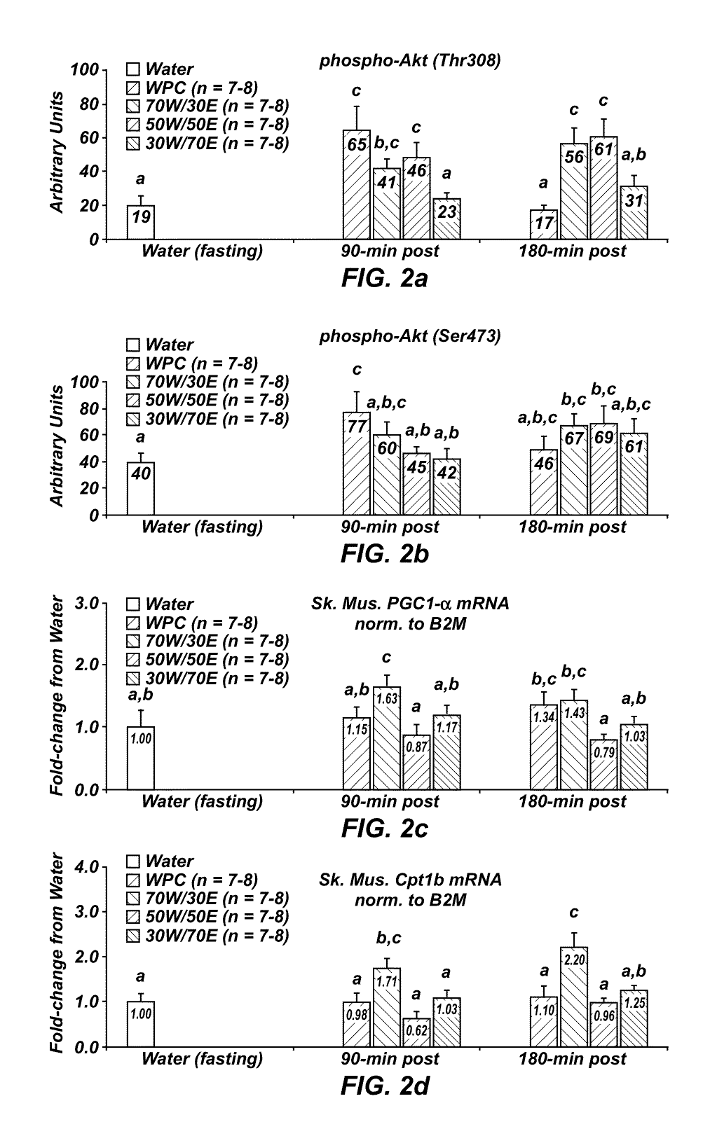

In FIG. 2, data are presented as means±standard error (water n=12-14 per bar, protein feeding groups n=6-8 per bar). One-way ANOVA with LSD post-hoc analyses were performed and significant between-feeding differences are represented with different superscript letters (p<0.05).

Immunoblotting results from mixed gastrocnemius muscles revealed: 1) rats fed WPC, 70W/30E and 50W/50E typically presented an increase in phospho-Akt (Thr308) compared to 30W/70E and fasted rats (p<0.05); 2) markers of mTOR signaling (p-mTOR Ser2481, p-p70s6k Thr389, p-rpS6 Ser235/236) were typically greater in rats fed 70W/30E and 50W/50E compared to WPC, 30W/70E and fasted rats (p<0.05); and 3) 180-min post-feeding muscle protein synthesis (MPS) analysis demonstrated that, compared to fasted rats, WPC feeding increased MPS by 94% (p<0.05), 70W/30E feeding increased MPS by 74% (p=0.065) and 50W/50E and 30W/70E feedings non-significantly increased MPS by 25% and 34%, respectively (p>0.05).

In summary, 70W/30E feeding elicits similar acute post-feeding responses to that of WPC. However, replacing hydrolyzed whey with a higher percentage of hydrolyzed egg albumin attenuated these responses. Feeding higher concentrations of hydrolyzed whey protein in combination with lower concentrations of hydrolyzed egg albumin elicits significant activation of anabolic biomarkers within skeletal muscle similar to those seen with acute whey protein concentrate post-feeding responses. Higher proportions of hydrolyzed whey had a greater impact on intramuscular metabolic markers relative to WPC and higher proportions of hydrolyzed egg albumin.

Further analysis follows:

Adult male Wistar rats (˜250 g) received one of five treatments and mixed gastroc muscle was analyzed 90 minutes post-feeding, as follows:

70% WPH/30% EPH (n=8)

30% WPH/70% EPH (n=8)

50% WPH/50% EPH (n=8)

WPC placebo (n=7)

Water placebo (n=6)

Additionally, adult male Wistar rats (˜250 g) received one of five treatments and mixed gastroc muscle was analyzed 180 minutes post-feeding, as follows:

70% WPH/30% EPH (n=7)

30% WPH/70% EPH (n=7)

50% WPH/50% EPH (n=7)

WPC placebo (n=7)

Water placebo (n=7)

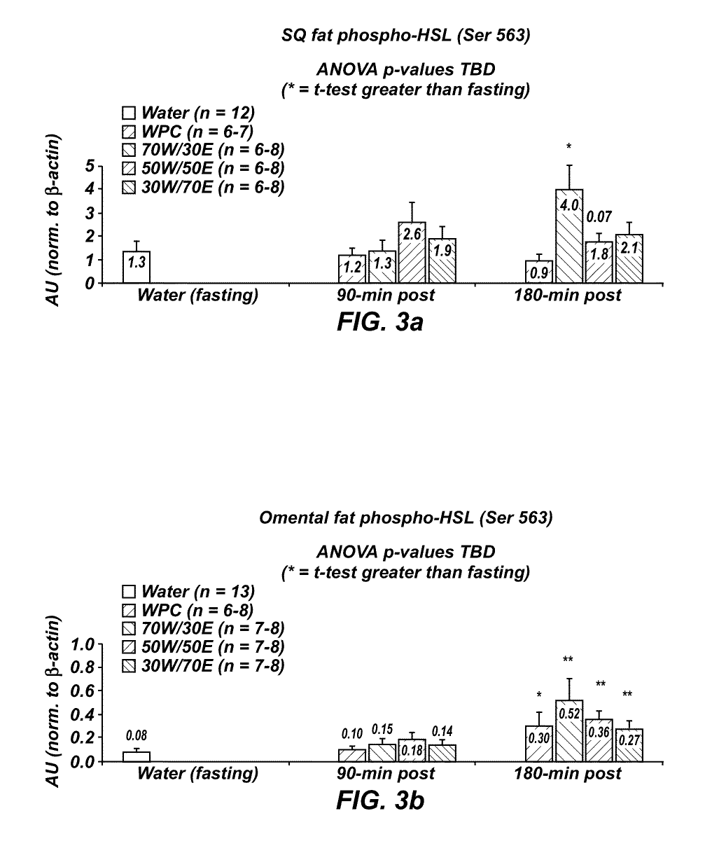

As shown by the data presented in FIGS. 3a and 3b, it is evident that 70% WPH/30% EPH, at 180 minutes post-feeding, has the greatest effects on subcutaneous fat and omental/visceral fat hormone-sensitive lipase action.

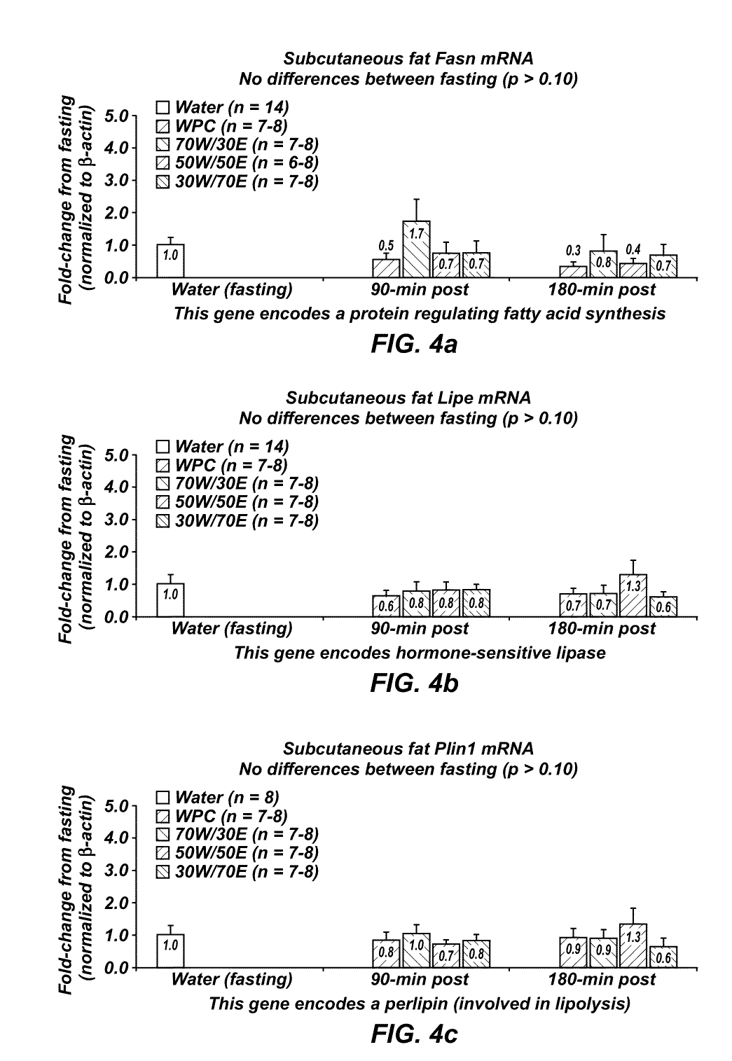

When looking at mRNA markers of lipogenesis (fatty acyl synthase) and lipolysis (Lipe and Plin1), as shown by the graphs of FIGS. 4a-4c, there were no statistical differences between the protein groups and fasting. The 90 minute 70% non-statistical rise in Fasn within the 70% WPH/30% EPH group may have been due to the superior insulinogenic response with hydrolyzed whey peptides, as insulin is known to promote fatty acid synthesis and fat storage within adipocytes.

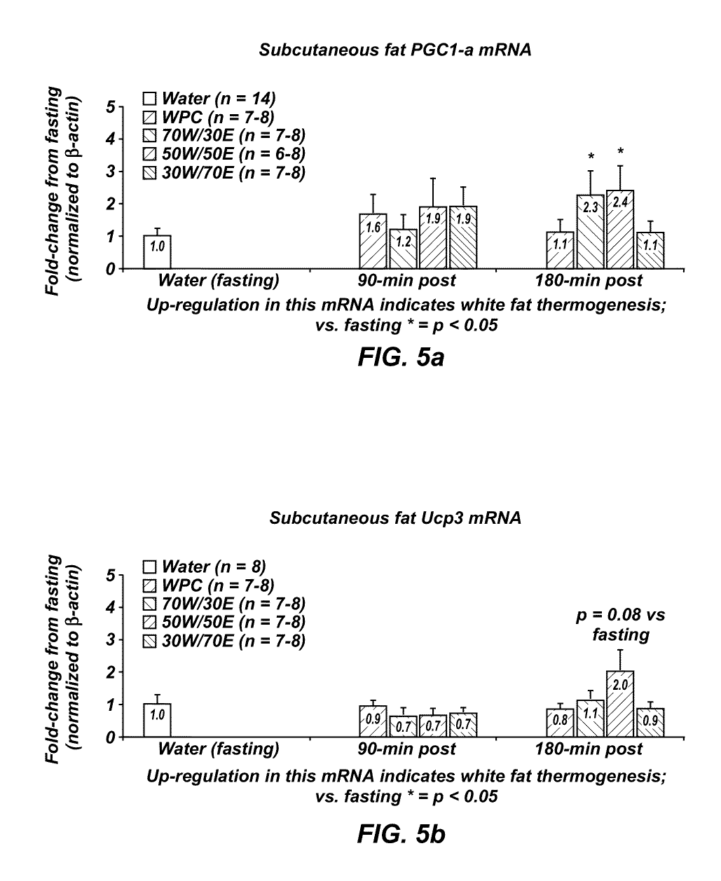

An increase in PGC1a and Ucp3 mRNAs would indicate that enhanced thermogenic processes are occurring; also termed as white adipose tissue “browning” or “briting.” At 180 minutes post-feeding, there is evidence to support this in the 70% WPH/30% EPH and/or the 50% WPH/50% EPH groups, as illustrated by the graphs of FIGS. 5a and 5b.

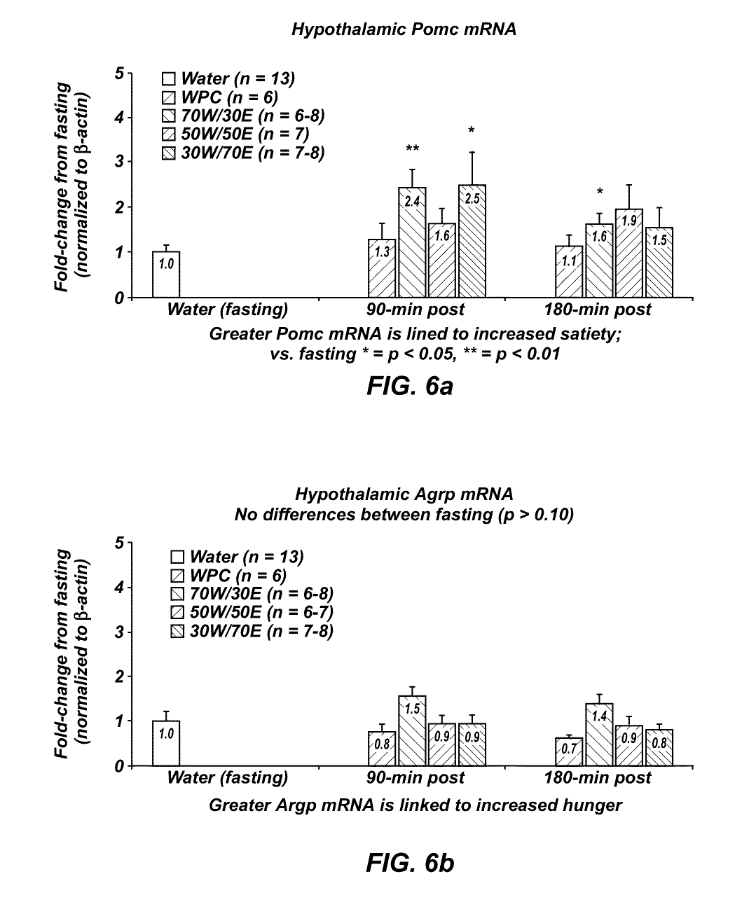

An increase in hypothalamic Pomc mRNA would indicate an increased satiety. Interestingly, as shown by the graphs of FIGS. 6a and 6b, all hydrolyzed protein sources seemed to provide this benefit at 90 minutes post-feeding and/or at 180 minutes post-feeding compared to WPC placebo and water placebo (i.e., fasting). None of the protein sources altered Agrp mRNA expression, which, when high, would indicate hunger.

### Adipose Tissue Lipolysis

We examined how gavage-feeding the following dietary proteins (10 human eq. g dissolved in 1 ml of water) acutely affected omental (OMAT) and inguinal/subcutaneous (SQ) adipose tissue lipolysis markers: a) 80% whey protein concentrate (WPC, n=15); b) 70% hydrolyzed whey+30% hydrolyzed egg albumin (70W/30E, n=15); c) 50W/50E (n=15); d) 30W/70E (n=15); and e) 1 ml of water with no protein (fasted, n=14).

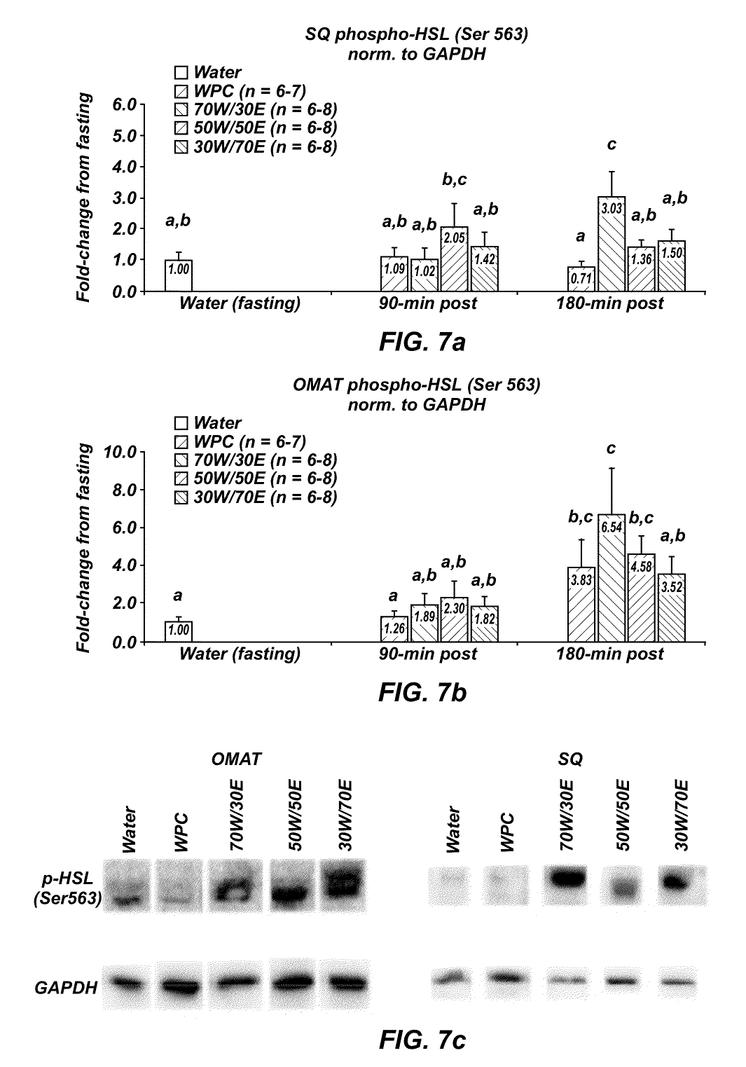

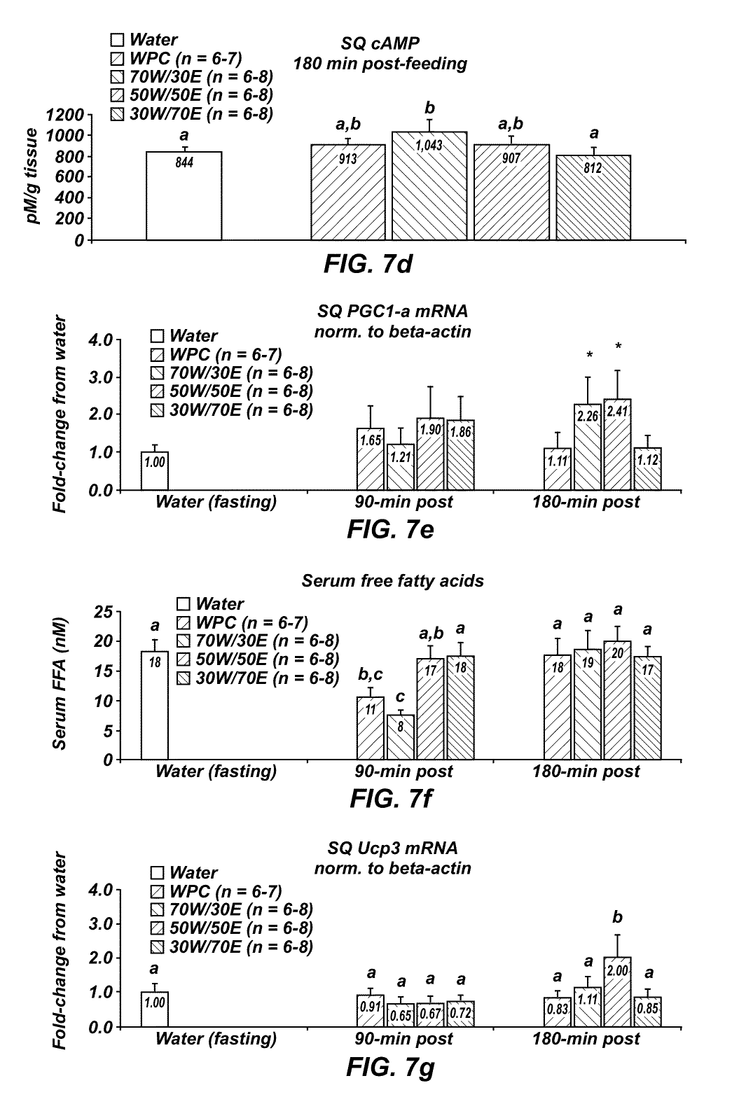

In FIG. 7, data are presented as means±standard error (water n=12-14 per bar, protein feeding groups n=6-8 per bar). One-way ANOVA with LSD post-hoc analyses were performed and significant between-feeding differences are represented with different superscript letters (p<0.05).

In FIGS. 7a-7d, 50W/50E caused the greatest 90 minutes post-feeding increase in SQ p-HSL, whereas 70W/30E caused the greatest increase in SQ p-HSL180 minutes post-feeding. 70W/30E also caused the greatest increase in SQ fat cAMP levels 180 minutes post-feeding.

FIG. 7b also shows that 70W/30E caused the greatest 180 minutes post-feed increase in OMAT p-HSL.

FIGS. 7e and 7g illustrate that 70W/30E and 50W/50E increased SQ PGC1-a mRNA compared to water 180 minutes post-feeding, and 50W/50E increased Ucp3 mRNA compared to all other groups at this time point.

FIG. 7f shows that WPC and 70W/30E caused an initial decrease in serum free fatty acids, but this decreased disappeared by 180 minutes post-feeding.

70W/30E feeding increased SQ fat phosphorylated hormone-sensitive lipase (p-HSL) 3.1-fold compared to fasting and 1.9-4.4-fold compared to all other test proteins 180 min post feeding (p<0.05). WPC, 70W/30E and 50W/50E feedings increased OMAT p-HSL 3.8-6.5-fold 180 min post-feeding compared to fasted rats (p<0.05). WPC and 70W/30E feedings depressed serum free fatty acids 90 min post-feeding compared to fasting and other test proteins (p<0.05), but this was normalized by 180 min post-feeding. Interestingly, 70W/30E and 50W/50E feedings tended to increase SQ fat PGC1-a mRNA 180 min post-feeding compared to fasting rats (2.3-2.4-fold, p<0.10), and 50W/50E significantly increased Ucp3 mRNA 180 min post-feeding compared to all other test proteins as well as fasting (˜2.0 fold, p<0.05).

In summary, despite a transient 90-min post feeding depression in serum FFAs with 70W/30E feeding, solutions with 50% and 70% hydrolyzed whey increased select tissue markers of lipolysis and thermogenesis 180 min post-feeding. Feeding higher concentrations of hydrolyzed whey protein (50-70%) increases markers of adipose tissue lipolysis and thermogenesis 180-min post-feeding compared to 30W/70W and whey protein concentrate feedings as well as fasting.

Further analysis follows:

Adult male Wistar rats (˜250 g) received one of five treatments and mixed gastroc muscle was analyzed 90 minutes post-feeding, as follows:

70% WPH/30% EPH (n=8)

30% WPH/70% EPH (n=8)

50% WPH/50% EPH (n=8)

WPC placebo (n=7)

Water placebo (n=6)

Additionally, adult male Wistar rats (˜250 g) received one of five treatments and mixed gastroc muscle was analyzed 180 minutes post-feeding, as follows:

70% WPH/30% EPH (n=7)

30% WPH/70% EPH (n=7)

50% WPH/50% EPH (n=7)

WPC placebo (n=7)

Water placebo (n=7)

At 90 minutes post-feeding, Akt phosyphorylation is higher with greater whey protein content (WPC>70W/30E=50W/50E>30W/70E), as shown in FIGS. 9a and 9b. This is an index of intramuscular insulin signaling. Since whey protein (native and hydrolyzed) are known insulin secretagogues, these findings are intuitive (i.e., less whey protein in solution yields less potential insulin secretion). These findings suggest that a whey protein bolus increases the phosphorylation of these markers.

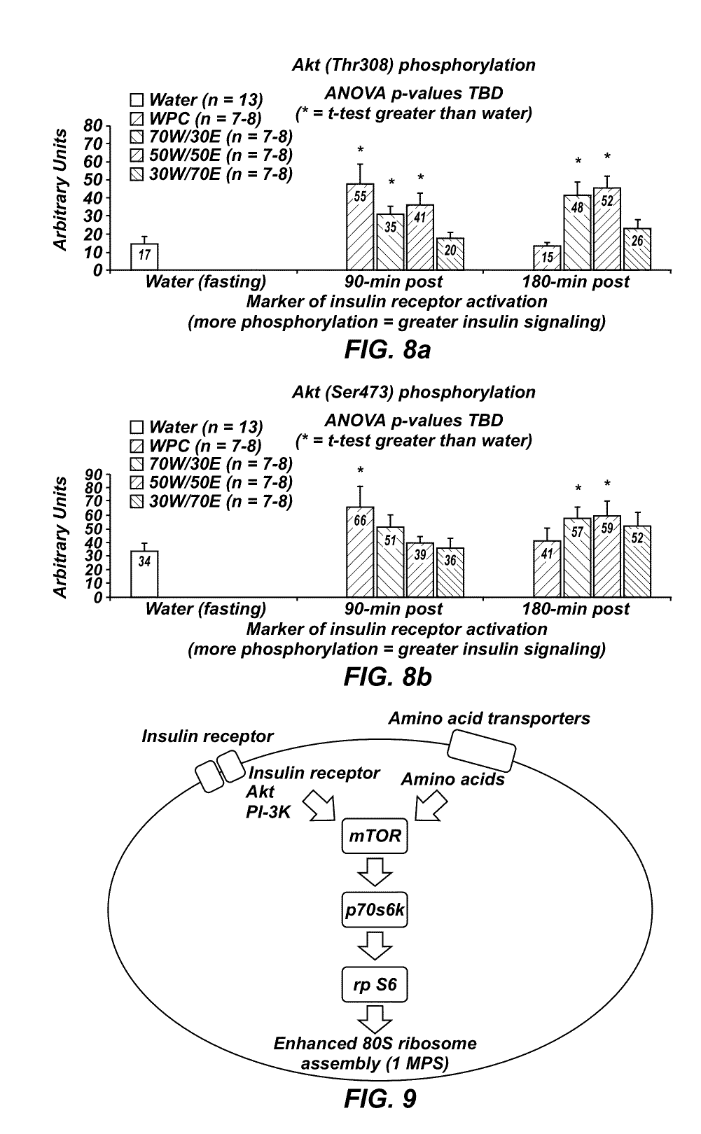

FIGS. 8a and 8b also show that Akt phosphorylation remained elevated 180 minutes post-feeding in the 70W/30E and 50W/50E compared to WPC. It has been recently demonstrated (Gaudel, et al. —PMID 23658425) that pancreatic beta cells treated in culture with WPC versus WPH demonstrated a greater insulin secretion profile with WPH. Hence, a prolonged Akt phosphorylation state following 70W/30E and 50W/50E compared to WPC feedings may reflect a more prolonged insulin signaling profile and continues to suggest that hydrolyzed whey may exhibit health benefits beyond muscle anabolism (i.e., glucose handling in diabetics).

As with p70s6k phosphorylation being elevated at 180 minutes post-feeding in the 70W/30E and 50W/50E groups (FIG. 8b), rpS6 phosphorylation is also elevated at 180 minutes past-feeding in the 70W/30E and 50W/50E groups. Patterns in rpS6 phosphorylation follow patterns in p70s6k phosphorylation and in mTOR phosphorylation.

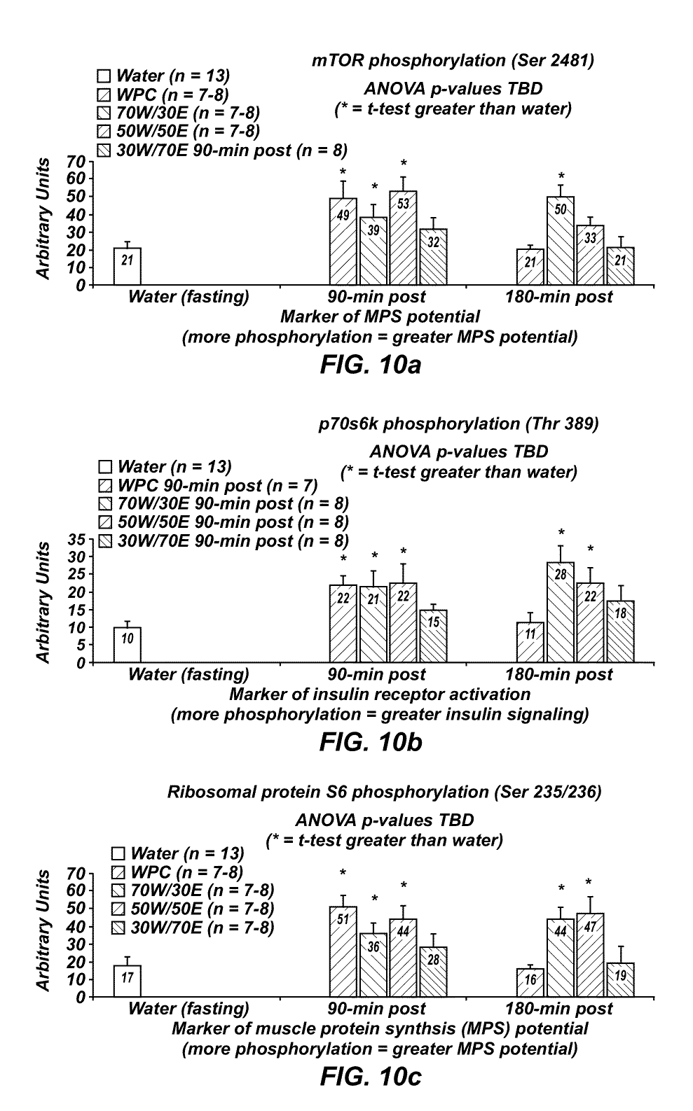

FIG. 9 is a diagram of the mTORC1 pathway. As shown in the graph of FIG. 10a, at 90 minutes post-feeding, mTOR phosphorylation is higher with greater whey protein content (WPC=70W/30E=50W/50E>30W/70E and fasting). These findings suggest that a whey protein bolus increases phosphorylation of mTOR.

Further, mTOR phosphorylation remained elevated at 180 minutes post-feeding in the 70W/30E group, but only in that group. This may be due to: a) the prolonged phosphorylation state following 70W/30E feedings, which, in turn, keeps mTOR activated; and/or b) the highest proportion of hydrolyzed whey, which could confer independent effects on mTOR activation beyond insulin-Akt signaling.

As illustrated by FIG. 10b, as with mTOR phosphorylation, p70s6k phosphorylation is higher with greater whey protein content at 90 minutes post-feeding (WPC=70W/30E=50W/50E>30W/70E and fasting). This is to be expected since p70s6k is activated downstream of mTOR. As with mTOR phosphorylation being elevated at 180 minutes post-feeding, but only in the 70W/30E group, p70s6k phosphorylation is higher with greater whey protein content at 180 minutes post-feeding in the 70W/30E and 50W/50E groups.

As with mTOR phosphorylation and p70s6k phosphorylation, ribosomal protein S6 (rpS6) phosphorylation is higher with greater whey protein content at 90 minutes post-feeding (WPC=70W/30E=50W/50E>30W/70E and fasting), as depicted by the graphs of FIG. 10c. These results were expected since rpS6 is activated downstream from mTOR and p70s6k. The same pattern of p70s6k phosphorylation being elevated at 180 minutes post-feeding in the 70W/30E and 50W/50E groups occurred rpS6 phosphorylation.

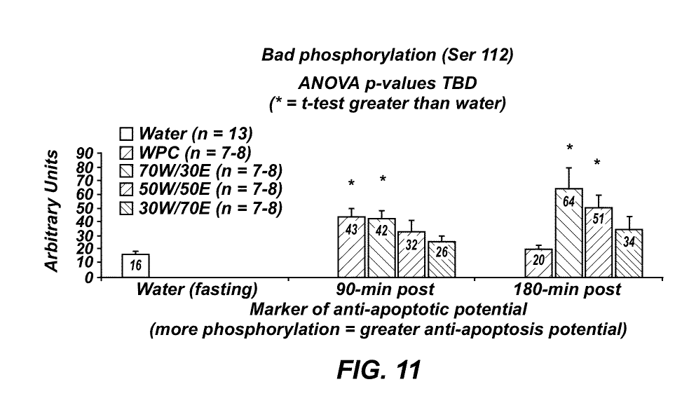

The protein array also provided a marker of apoptosis. The protein BAD promotes apoptosis through mitochondrial membrane pore permeabilization and is inactivated through phosphorylation. We find that WPC and 70W/30E protein feedings increases a marker of BAD inactivation 90 minutes post-feeding, as shown in FIG. 11. Remarkably, this marker remains elevated in 70W/30E protein feedings 180 minutes post-feeding and rises in the 50W/50E group at this point in time as well.

Although the foregoing disclosure provides many specifics, these should not be construed as limiting the scope of any of the ensuing claims. Other embodiments may be devised which do not depart from the scopes of the claims. Features from different embodiments may be employed in combination. The scope of each claim is, therefore, indicated and limited only by its plain language and the full scope of available legal equivalents to its elements.

What is claimed:

1. A protein supplement, including:
an animal-based protein hydrolysate; and
an immune modulator.

2. The protein supplement of claim 1, wherein the immune modulator is from a source other than the animal-based protein hydrolysate.

3. The protein supplement of claim 1, wherein the animal-based protein hydrolysate comprises a hydrolysate of milk proteins.

4. The protein supplement of claim 3, wherein the animal-based protein hydrolysate comprises a hydrolysate of whey proteins.

5. The protein supplement of claim 1, wherein the animal-based protein-hydrolysate comprises a hydrolysate of egg proteins.

7. The protein supplement of claim 1, wherein the immune modulator comprises nanofraction immune modulators.

8. The protein supplement of claim 7, wherein the immune modulator has an upper molecular weight cutoff of about 5 KD or less.

9. The protein supplement of claim 8, wherein the immune modulator substantially lacks transfer factor.

10. The protein supplement of claim 1, wherein the immune modulator comprises transfer factor.

11. The protein supplement of claim 10, wherein the immune modulator has an upper molecular weight cutoff of about 12 KD or less.

12. The protein supplement of claim 10, wherein the immune modulator has an upper molecular weight cutoff of about 10 KD or less.

13. A protein supplement, including:
a whey hydrolysate; and
a fraction of at least one of colostrum and egg, each fraction having an upper molecular weight cutoff of about 12 KD or less.

14. The protein supplement of claim 13, consisting essentially of the whey hydrolysate and the fraction of at least one of colostrum and egg.

15. The protein supplement of claim 13, consisting of the whey hydrolysate and the fraction at least one of colostrum and egg.

16. The protein supplement of claim 13, wherein each fraction has a molecular weight cutoff of about 10 KD or less.

17. The protein supplement of claim 13, wherein each fraction has a molecular weight cutoff of about 5 KD or less.

18. The protein supplement of claim 13, wherein the whey hydrolysate is a hydrolysate of standard whey.

19. The protein supplement of claim 13, wherein the whey hydrolysate is a hydrolysate of a whey protein concentrate.

20. The protein supplement of claim 13, wherein the whey hydrolysate is a hydrolysate of a whey protein isolate.

21. A protein supplement, including:
egg protein from avian eggs;
milk protein.

22. The protein supplement of claim 21, wherein the egg protein comprises albumin.

23. The protein supplement of claim 21, wherein the egg protein comprises a whole egg hydrolysate.

24. The protein supplement of claim 21, wherein the milk protein comprises whey protein.

25. The protein supplement of claim 24, wherein the whey protein comprises a whey protein concentrate.

26. The protein supplement of claim 24, wherein the whey protein comprises a whey protein isolate.

27. The protein supplement of claim 24, wherein the whey protein comprises a hydrolysate of whey, a hydrolysate of a whey protein concentrate or a hydrolysate of a whey protein isolate.

28. The protein supplement of claim 21, wherein the milk protein comprises a milk protein hydrolysate.

29. The protein supplement of claim 21, consisting essentially of the egg protein and the milk protein.

30. The protein supplement of claim 21, further comprising:
an immune modulator.

31. The protein supplement of claim 30, wherein the immune modulator includes transfer factor.

32. The protein supplement of claim 31, wherein at least about 80% of all proteins of the immune modulator are proteins having molecular weights of less than 5,000 Da.

33. The protein supplement of claim 32, wherein at least about 50% of all proteins of the immune modulator are proteins having molecular weights of less than 3,000 Da.

34. The protein supplement of claim 33, wherein the immune modulator comprises colostrum or a fraction of colostrum.

35. The protein supplement of claim 34, further comprising: another immune modulator.

36. The protein supplement of claim 35, wherein the another immune modulator comprises avian transfer factor.

37. The protein supplement of claim 36, consisting essentially of the egg protein, the milk protein, the immune modulator and the another immune modulator.

38. The protein supplement of claim 21, lacking protein from a plant source.

39. The protein supplement of claim 21, lacking soy protein.

40. The protein supplement of claim 21, wherein:
the milk protein makes up about 70% of a combined weight of the milk protein and the egg protein; and
the egg protein makes up about 30% of the combined weight.

41. The protein supplement of claim 21, wherein:
the milk protein makes up about 60% of a combined weight of the milk protein and the egg protein; and
the egg protein makes up about 40% of the combined weight.

42. The protein supplement of claim 21, wherein:
the milk protein makes up about 50% of a combined weight of the milk protein and the egg protein; and
the egg protein makes up about 50% of the combined weight.

43. The protein supplement of claim 21, wherein:
the milk protein makes up about 40% of a combined weight of the milk protein and the egg protein; and
the egg protein makes up about 60% of the combined weight.

44. The protein supplement of claim 21, wherein:
the milk protein makes up about 30% of a combined weight of the milk protein and the egg protein; and
the egg protein makes up about 70% of the combined weight.

45. A food product including the protein supplement of claim 1.

46. The food product of claim 45, comprising an energy bar.

47. A baby formula including the protein supplement of claim 1.

48. A method for reducing inflammation in a body of a subject, comprising administering a composition according to claim 1 to the subject.

49. A method for supplementing a diet of a subject, comprising replacing at least a portion of the subject's diet with a composition according to claim 1.

50. The method of claim 49, wherein administering comprises administering the composition in place of breakfast.

51. The method of claim 49, comprising reducing body fat in the subject's body.

52. A method for increasing at least one of muscle mass and muscle strength of a subject, comprising administering a composition according to claim 1 to the subject.

53. The method of claim 52, wherein administering comprises administering the composition to the subject within an hour of increased physical activity.

54. A method for reducing muscle catabolism in a subject, comprising administering a composition according to claim 1 to the subject.
